(19) United States
(12) Patent Application Publication
(10) Pub. No.: US 2015/0190055 A1
(43) Pub. Date: Jul. 9, 2015

(54) MAGNETIC RESONANCE IMAGING APPARATUS CAPABLE OF ACQUIRING SELECTIVE GRAY MATTER IMAGE, AND MAGNETIC RESONANCE IMAGE USING SAME

(71) Applicant: Korea University Research and Business Foundation, Seoul (KR)

(72) Inventors: Jaeseok Park, Seoul (KR); Hyunyeol Lee, Seoul (KR)

(21) Appl. No.: 14/390,109

(22) PCT Filed: Apr. 4, 2013
(86) PCT No.: PCT/KR2013/002807

§ 371 Date: Jan. 19, 2015

(30) Foreign Application Priority Data
Apr. 5, 2012 (KR) 10-2012-0035696

(51) Int. Cl. A61B 5/00; A61B 5/055; G06T 11/00

(52) U.S. Cl. CPC A61B 5/0042; G06T 11/008; A61B 5/055; G06T 2207/10088; G06T 2207/20216; G06T 2207/30016; A61B 2576/026

(57) ABSTRACT

There is provided a magnetic resonance imaging apparatus comprising: an inversion pulse generating unit that applies an inversion pulse to a living body to suppress a white matter image signal; an excitation pulse generating unit that applies an RF excitation pulse to the living body after inversion time from the inversion pulse so as to excite magnetization; an image signal receiving unit that acquires first and second final image signals from first and second echo trains in an RF refocusing pulse train, respectively; and an image generating unit that generates a gray matter image from the first and second final image signals.

### TECHNICAL FIELD

The embodiments described herein pertain generally to a magnetic resonance imaging apparatus and a method for acquiring a magnetic resonance image by using the magnetic resonance imaging apparatus, in particular, a magnetic resonance imaging apparatus, which is capable of acquiring a selective gray matter image, and a method for acquiring a magnetic resonance image.

### BACKGROUND

In recent, there have been increasing cases of acquiring an image of a human body in a lateral direction, a longitudinal direction, a diagonal direction and other directions by using a magnetic resonance imaging (MRI) apparatus, and examining and diagnosing the state of a person to be examined through the image.

A technique of acquiring an image having high resolution and contrast is being researched for more exact diagnosis, and especially, a technique of selectively displaying an image related to interested information within a final image acquired in the magnetic resonance image apparatus, e.g., only gray matter information, is being currently researched.

The technique of selectively acquiring a gray matter image includes a double inversion recovery technique, which is described below with reference to FIG. 1.

FIG. 1 shows a process for acquiring a selective gray matter image according to the double inversion recovery technique applied to the magnetic resonance imaging apparatus.

In the double inversion recovery technique, two (2) inversion pulses are applied prior to each pulse train for acquisition of data. A first inversion pulse is applied with long inversion time to suppress a signal of cerebrospinal fluid, long inversion time is required, while a second inversion pulse is applied short inversion time to suppress a signal of a white matter. Such two (2) inversion pulses are applied each time a pulse train is repeated, and only a gray matter signal is residual during the period of time for data acquisition so that a gray matter image is selectively acquired.

This technique is introduced in the journal, Pouwels P. et al., “Human Gray Matter: Feasibility of Single-Slab 3D Double Inversion-Recovery High-Spatial-Resolution MR Imaging,” Radiology, 2006; 241: 873-879.

However, since the double inversion recovery technique uses the two (2) inversion pulses and the long and short inversion time, it has been problematic in that it requires significantly long time to acquire an image, and a signal to noise ratio (SNR) for the acquired gray matter image is low due to a reduction of the gray matter signal residual during the time of data acquisition for restoration of a final image. Due to these problems, the conventional double inversion recovery technique has had difficulty in acquiring a high-resolution image, especially, the gray matter image at a high speed.

### DISCLOSURE OF THE INVENTION

### Problems to be Solved by the Invention

In view of the foregoing problems, example embodiments provide a magnetic resonance imaging apparatus, which applies only one inversion pulse to suppress a white matter image signal, and requires short inversion time so as to reduce time required to acquire an image and selectively acquire a cerebral gray matter image having a high SNR at a high speed.

Also, example embodiments provide a magnetic resonance imaging apparatus, which independently processes image signals acquired from two (2) consecutive echo trains for encoding an image, respectively, so as to selectively acquire a high-resolution cerebral gray matter image having a high SNR.

### Means for Solving the Problems

In accordance with a first aspect (example embodiment) of the present disclosure, there is provided a magnetic resonance imaging apparatus comprising: an inversion pulse generating unit that applies an inversion pulse to a living body to suppress a white matter image signal; an excitation pulse generating unit that applies an RF excitation pulse to the living body after inversion time from the inversion pulse so as to excite magnetization; an image signal receiving unit that acquires first and second final image signals from first and second echo trains in an RF refocusing pulse train, respectively, after the application of the RF excitation pulse; and an image generating unit that generates a gray matter image from the first and second final image signals, wherein the first and second final image signals are formed by first and second image signals acquired once or more from the first and second echo trains, respectively.

Especially, the image signal receiving unit may independently rearrange the first and second final image signals in first and second K-spaces, respectively, the first final image signal may be rearranged in the direction from the center of the first K-space toward the peripheral side thereof, and the second final image signal may be rearranged in the direction from the peripheral side of the second K-space toward the center thereof.

Especially, the first final image signal may comprise a gray matter image signal and a first cerebrospinal fluid image signal, and the second final image signal may comprise a second cerebrospinal fluid image signal.

Herein, flip angles in the RF refocusing pulse train may be set to make intensity of the first cerebrospinal fluid image signal and intensity of the second cerebrospinal fluid image signal identical to each other.

In accordance with a first aspect (example embodiment) of the present disclosure, there is provided a magnetic resonance imaging apparatus comprising: an inversion pulse generating unit that applies an inversion pulse to a living body to suppress a white matter image signal; an excitation pulse generating unit that applies an RF excitation pulse to the living body after inversion time from the inversion pulse so as to excite magnetization; an image signal receiving unit that acquires first and second final image signals from first and second echo trains in an RF refocusing pulse train, respectively, after the application of the RF excitation pulse; and an image generating unit that generates a gray matter image from the first and second final image signals, wherein the first and second final image signals are formed by first and second image signals acquired once or more from the first and second echo trains, respectively.

Especially, the image signal receiving unit may independently rearrange the first and second final image signals in first and second K-spaces, respectively, the first final image signal may be rearranged in the direction from the center of the first K-space toward the peripheral side thereof, and the second final image signal may be rearranged in the direction from the peripheral side of the second K-space toward the center thereof.

Especially, the first final image signal may comprise a gray matter image signal and a first cerebrospinal fluid image signal, and the second final image signal may comprise a second cerebrospinal fluid image signal.

Herein, flip angles in the RF refocusing pulse train may be set to make intensity of the first cerebrospinal fluid image signal and intensity of the second cerebrospinal fluid image signal identical to each other.

Herein, flip angles in the RF refocusing pulse train may be set to enable intensity of the gray matter image signal to have a predetermined value or more.

In accordance with a second aspect (another example embodiment) of the present disclosure, there is provided a method for acquiring a magnetic resonance image, comprising: (a) applying an inversion pulse to a living body to suppress a white matter image signal; (b) applying an RF excitation pulse to the living body after inversion time caused by the inversion pulse to excite magnetization; (c) acquiring first and second image signals from first and second echo trains within an RF refocusing pulse train, respectively, after the application of the RF excitation pulse; (d) implementing steps (a) to (c) above once or more to form first and second final image signals by the first and second image signals; and (e) acquiring a gray matter image from the first and second final image signals.

Herein, the method for acquiring a magnetic resonance image may further comprise preparing longitudinal magnetization in the living body prior to the application of the first inversion pulse in the step (a) above.

Especially, the step (e) above may comprise: generating first and second reconstructed images from the first and second final image signals; and implementing weighted average processing for the first and second reconstructed images to generate a gray matter image.

### Effect of the Invention

In accordance with the example embodiments, the magnetic resonance imaging apparatus and the method for acquiring an image by using the apparatus are advantageous in that they can significantly reduce total imaging time, compared to a conventional technique for acquiring a selective gray matter image, by applying only one inversion pulse with short inversion time for suppressing a white matter image signal. Thus, they fundamentally eliminate the necessity for long inversion time, and simultaneously, acquire a high-resolution gray matter image having high signal intensity.

Furthermore, in accordance with the example embodiments, the magnetic resonance imaging apparatus and the method for acquiring an image by using the apparatus are advantageous in that with only one inversion pulse two final image signals are acquired from two (2) consecutive echo trains in an RF refocusing pulse train, respectively, and are independently processed, so that the signal to noise ratio loss is minimized, and thus, a user can acquire a selective gray matter image having high resolution at a higher speed.

### BRIEF DESCRIPTION OF THE DRAWINGS

FIG. 1 shows a process for acquiring a selective gray matter image according to a double inversion recovery technology applied to a magnetic resonance imaging apparatus.

FIG. 2 is a block configuration diagram showing a whole magnetic resonance imaging apparatus in accordance with an example embodiment.

FIG. 3 is a block configuration diagram showing a magnified view of partial components of FIG. 2.

FIG. 4 and FIG. 5 show setting flip angles in an RF refocusing pulse train.

FIG. 6 shows a technique of rearranging first and second final image signals acquired by an image signal receiving unit of FIG. 3 in an independent K-space.

FIG. 7 is a block configuration diagram specifically showing an image generating unit of FIG. 3.

FIG. 8 shows a selective gray matter image acquired by using the magnetic resonance image apparatus in accordance with an example embodiment.

FIG. 9 shows a degree of change in an intensity of an echo signal depending on preparation of magnetization.

FIG. 10 shows a process for acquiring a selective gray matter image by the magnetic resonance imaging apparatus in accordance with an example embodiment.

FIG. 11 is a flow chart showing a method for acquiring a magnetic resonance image in accordance with an example embodiment.

FIG. 12 is a sequence view showing a method for acquiring a magnetic resonance image in accordance with another example embodiment.

FIG. 13 is a sequence view specifically showing S1260 of FIG. 12.

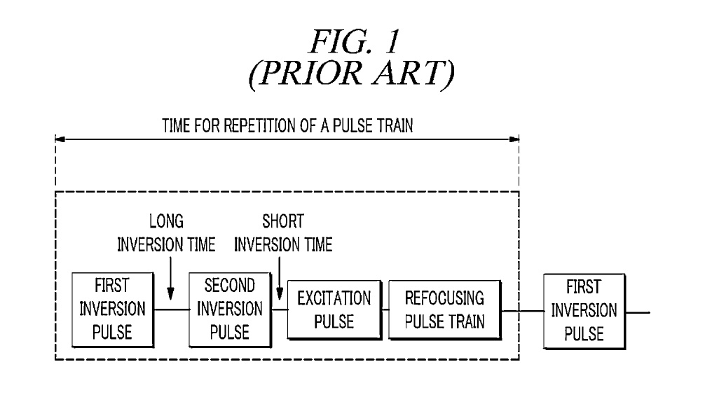

### DETAILED DESCRIPTION

Hereinafter, example embodiments will be described in detail with reference to the accompanying drawings so that inventive concept may be readily implemented by those skilled in the art. However, it is to be noted that the present disclosure is not limited to the example embodiments, but can be realized in various other ways. In the drawings, certain parts not directly relevant to the description are omitted to enhance the clarity of the drawings, and like reference numerals denote like parts throughout the whole document.

Throughout the whole document, the terms “connected to” or “coupled to” are used to designate a connection or coupling of one element to another element and include both a case where an element is “directly connected or coupled to” another element and a case where an element is “electronically connected or coupled to” another element via still another element. Further, the term “comprises or includes” and/or “comprising or including” means that one or more other components, steps, operations, and/or the existence or addition of elements are not excluded in addition to the described components, steps, operations and/or elements.

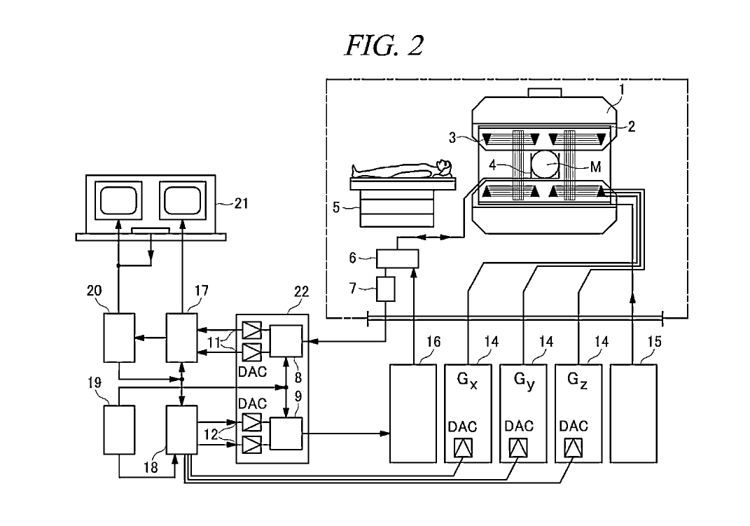

FIG. 2 is a block configuration diagram showing a whole magnetic resonance imaging apparatus in accordance with an example embodiment. Here, the magnetic resonance imaging (MRI) apparatus refers to an apparatus using a magnetic field harmless to a human body and specific ionization radiation (radio high frequency) to make images for the physical principle of the nuclear magnetic resonance (NMR), and has a structure substantially identical to that of a conventional tomography device.

A main magnet 1 generates a strong magnetic field in a certain size to polarize or arrange nuclear spins within an area of an object to be examined, for example, like part of a human body to be examined. High homogeneity of the main magnet necessary for measurement of nuclear spin resonance is determined within a spherical measurement space (M), and part of a human body to be examined enters into the measurement space M. In this case, a shim plate made of a so-called ferromagnetic material is provided at a position appropriate for meeting the homogeneity requirement, and especially, eliminating time-invariable operations. Time-variable operations are eliminated by a shim coil 2 driven by a shim supply 15.

A cylindrical slant coil system 3 consisting of three (3) partial wires is inserted into the main magnet 1. Each of the partial wires receives a current from an amplifier 14 to generate a linear slant field in an individual direction of a parallel coordinate. Here, a first partial wire of the slant field system 3 generates slant Gx in the direction of x, a second partial wire of the slant field system 3 generates slant Gy in the direction of y, and a third partial wire of the slant field system 3 generates slant Gz in the direction of z. Each of the amplifiers 14 has a digital-analogue converter, which is controlled by a sequence control system 18 to generate a slant pulse exactly on time.

A high frequency antenna 4 is provided within the slant field system 3, whereby the high frequency antenna 4 excites a nuclear and converts a high frequency pulse emitted by a high frequency power amplifier 16 into an alternating field in order to arrange nuclear spins in an object to be examined or an area thereof. The alternating field emitted from the nuclear spins revolving by the high frequency antenna 4, i.e., a nuclear spin echo signal caused by a pulse sequence generally consisting of at least one high frequency pulse and at least one slant pulse is converted into voltage, and the voltage is supplied by an amplifier 7 to a high frequency receiving channel 8 of a high frequency system 22.

In addition, the high frequency system 22 includes a transmitting channel 9, and a high frequency pulse for exciting magnetic nuclear resonance is generated within the transmitting channel 9. In this case, an individual high frequency pulse is marked with a series of complex numbers in a digital manner within the sequence control system 18 according to a pulse sequence preset by an installed computer 20. The series of complex numbers have real and imaginary parts, which pass by their respective input ports 12 and are supplied to the digital-analogue converter connected to the high frequency system 22, so as to be supplied from the digital-analogue converter to the transmitting channel 9. In this case, the pulse sequence within the transmitting channel 9 is modulated into a high frequency carrier signal, and a basic frequency of the high frequency carrier signal corresponds to a resonance frequency of the nuclear spins present within the measurement space.

In this case, in the connection between the slant field system 3 and the high frequency system 22, conversion from a transmitting operation by the transmitting channel 9 into a receiving operation by the high frequency receiving channel 8 is implemented by a duplexer 6.

The high frequency antenna 4 radiates the high frequency pulse for exciting the nuclear spins into the measurement space M and implements sampling of an echo signal appearing as a result of the radiation. A nuclear resonance signal acquired in correspondence to the sampling is phase-sensitively decoded within the receiving channel 8 of the high frequency system 22, and converted into real and imaginary parts of the measured signal by the individual analogue-digital converter. An image processing device 17 processes signal data, which pass by their respective output ports 11 and are supplied to the image processing device 17, to reconstruct the data to be one image.

Management of measured data, image data and a control program is implemented by the installed computer 20, and the sequence control system 18 controls generation of a certain individual pulse sequence and sampling of a corresponding K-space through presetting by the control program.

In this case, the sequence control system 18 controls slant conversion according to exact time, radiation of a high frequency pulse having a preset phase and amplitude, and reception of a nuclear resonance signal, and a sound synthesizer 19 provides a time base for the high frequency system 22 and the sequence control system 18. Selection of a proper control program for generating a nuclear spin image is implemented by one keypad of a generated nuclear spin image and a terminal 21 having at least one display.

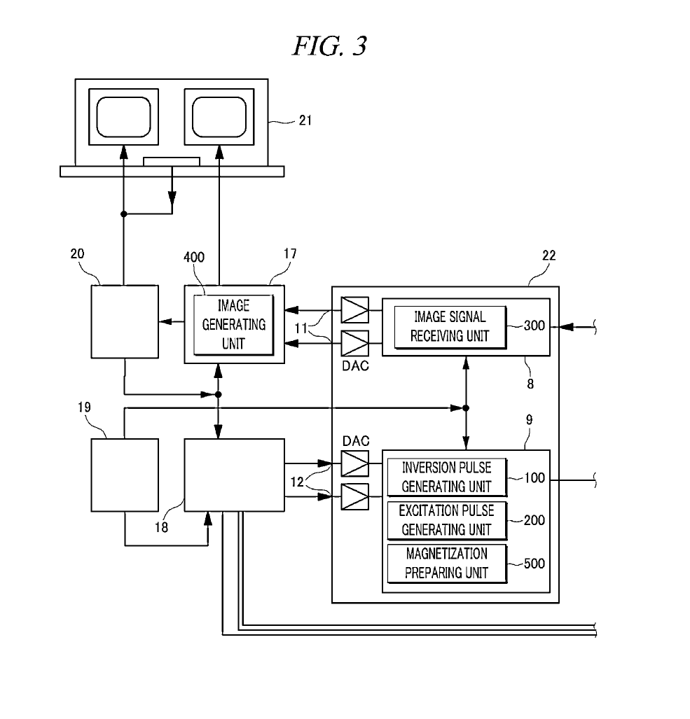

Hereinafter, detailed configuration of the magnetic resonance imaging apparatus in accordance with an example embodiment is described with reference to FIG. 3. FIG. 3 is a block configuration diagram showing a magnified view of partial components of FIG. 2.

The magnetic resonance imaging apparatus in accordance with an example embodiment includes: an inversion pulse generating unit 100 for applying an inversion pulse to a living body to suppress a white matter image signal; an excitation pulse generating unit 200 for applying an RF excitation pulse after inversion time from the inversion pulse so as to excite magnetization to the living body; an image signal receiving unit 300 for acquiring first and second final image signals from first and second echo trains in an RF refocusing pulse train, respectively, after the application of the RF excitation pulse; and an image generating unit 400 for generating a gray matter image from the first and second final image signals.

The inversion pulse generating unit 100 generates a 180° inversion pulse to be applied to a living body, and the inversion pulse operates such that a positive (+) sign of a magnetized component within the living body, to which the inversion pulse has been applied, is changed into a negative (−) sign. A final image in which a white matter signal is selectively suppressed can be acquired by using the characteristic of tissues within the living body, which is magnetically recovered from the negative to the positive according to a T1 relaxation phenomenon during the inversion time after the inversion pulse, and the inversion pulse generating unit 100 may be provided in or combined to the transmitting channel 9 within the high frequency system 22 as illustrated in FIG. 3.

The excitation pulse generating unit 200 generates an RF excitation pulse to be applied to the area, to which the inversion pulse has been applied by the inversion pulse generating unit 100 as described above, and applies the RF excitation pulse after the inversion time from the inversion pulse so as to excite magnetization within the living body and obtain an image signal to be encoded. Like the above-described inversion pulse generating unit 100, the excitation pulse generating unit 200 may also be provided in or combined to the transmitting channel 9 within the high-frequency system 22.

The image signal receiving unit 300 acquires first and second final image signals generated by applying a multiple number of refocusing pulses after the RF excitation pulse, and acquires the first final image signal, which includes a gray matter image signal having information related to a gray matter image and a first cerebrospinal fluid image signal, from a first echo train, and the second final image signal, which includes a second cerebrospinal fluid image signal, from a second echo train, to independently encode the first and second final image signals.

Here, the first and second echo trains mean consecutive pulse trains present within an RF refocusing pulse train, and the first and second final image signals are formed by first and second image signals acquired once or more from the first and second echo trains, respectively. Additionally, the image signal receiving unit 300 may be provided in or combined to the high frequency receiving channel 8 within the high frequency system 22 as illustrated in FIG. 3.

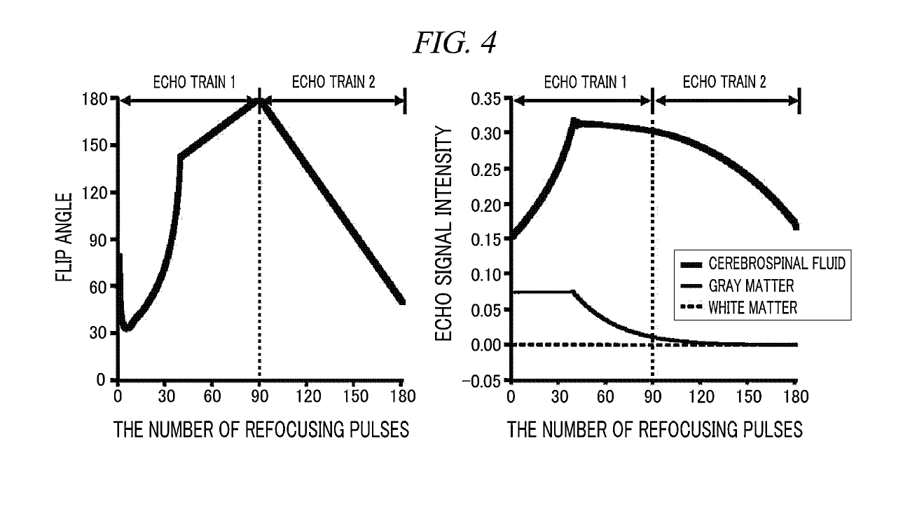

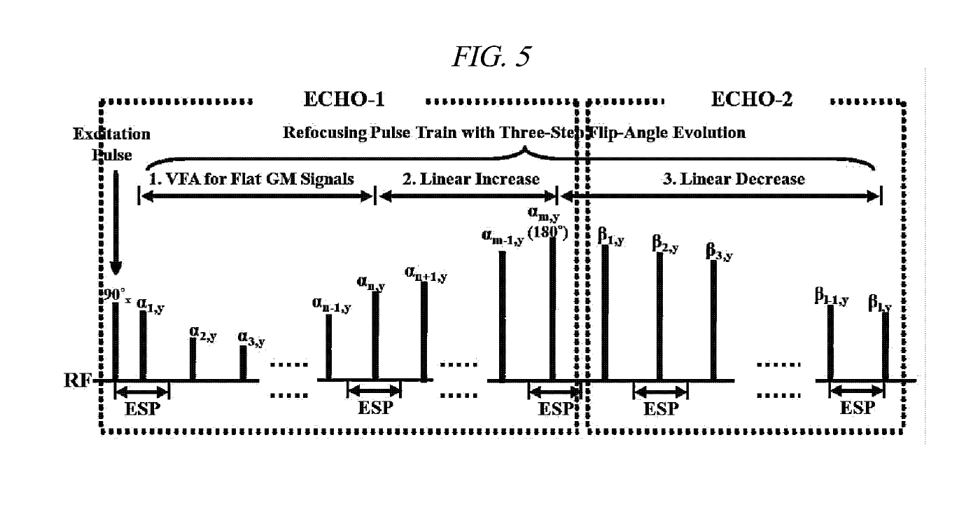

In this case, in order to selectively generate a high-resolution gray matter image in an image generating unit 400, which is described later, a user needs to prudently set or design flip angles in the RF refocusing pulse train, and setting the flip angle is described with reference to FIG. 4 and FIG. 5. FIG. 4 and FIG. 5 show setting the flip angles in the RF refocusing pulse train.

The flip angle means an angle, at which longitudinal magnetization having low energy and directed upwardly (parallel) absorbs energy to be changed into high-energy downward (anti-parallel, the excited state of spin orientations) magnetization.

Instead of eliminating the inversion pulse applied to suppress the cerebrospinal fluid image signal and the long inversion time in the conventional double inversion recovery technique, example embodiments selectively acquire a gray matter image while suppressing the cerebrospinal fluid image signal by using the first cerebrospinal fluid image signal included in the first final image signal and the second cerebrospinal fluid image signal included in the second final image signal. Accordingly, noise amplification can be minimized when the intensity of the gray matter image signal included in the first final image signal is the highest, while the first and second cerebrospinal fluid image signals have substantially identical or similar intensity, and the user can acquire a high-resolution final image having no artifacts.

Accordingly, it is preferable to set the flip angles in the RF refocusing pulse train such that the intensity of the first cerebrospinal fluid image signal and the intensity of the second cerebrospinal fluid image signal are identical to each other, and the intensity of the gray matter image signal can reach a certain level having a preset value or higher.

As in the example embodiment illustrated in FIG. 4 for setting the flip angles in the RF refocusing pulse train, the first echo train, from which the first image signal or the first final image signal is acquired, may be divided into two (2) sections, of which the front section is set to have a variable flip angle (VFA), and the rear section is set to have a linear increase in flip angles, and the second echo train, from which the second image signal or the second final image signal is acquired, may be set to have a linear decrease in flip angles.

The left graph of FIG. 5 illustrates another example for setting the flip angles of the RF refocusing pulse train, and the right graph of FIG. 5 illustrates results from numerical simulations for signal evolution along the echo train for the white matter, gray matter and cerebrospinal fluid in the case where the flip angles in the RF refocusing pulse train are set as in the example embodiment.

To specifically describe results of the simulated experiment, the flip angle of the initial part of the first echo train is calculated and set such that the signal of the gray matter is evenly evolved with a certain intensity, and thereby, preventing generation of artifacts resulting from signal modulation. The refocusing flip angles of the part ranging from the middle to the end of the first echo train are set to gradually increase up to 180° so as to increase the signal intensity of the gray matter to the maximum. In case of the second echo train, the refocusing flip angles are set to gradually decrease from 180° such that the cerebrospinal fluid signal intensity in the second echo train is substantially identical to the cerebrospinal fluid signal intensity in the first echo train.

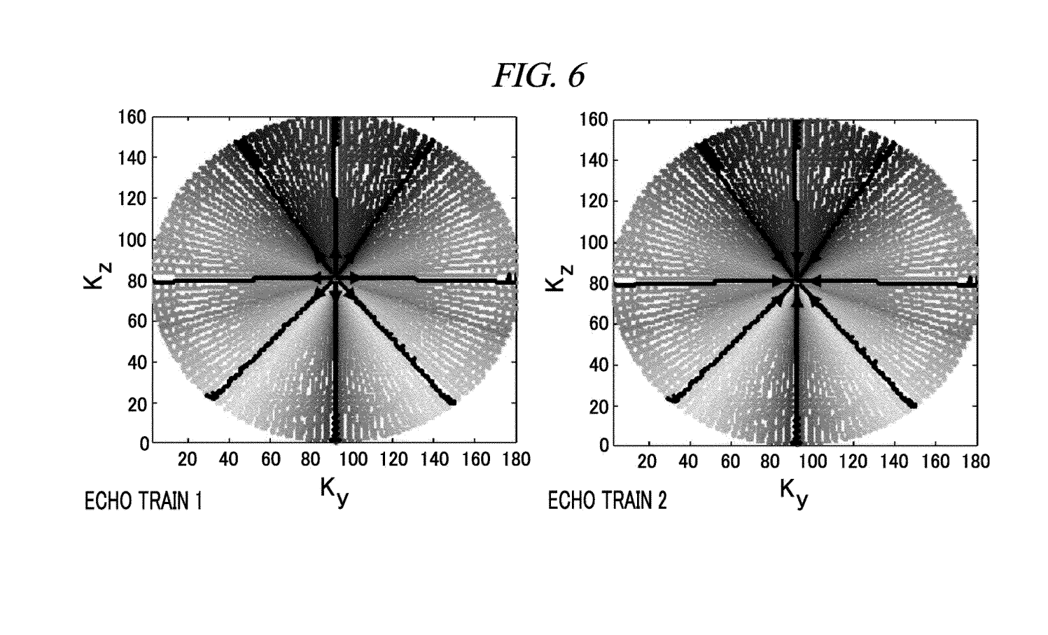

In addition, the image signal receiving unit 300 may rearrange the acquired first and second final image signals as shown in FIG. 6. FIG. 6 shows a technique for rearranging the first and second final image signals acquired by the image signal receiving unit of FIG. 3 in two independent K-spaces, and the first and second final image signals may be independently subject to sampling in two K-spaces.

With respect to examples for the technique of rearranging the image signals by the image signal receiving unit 300, the image signal receiving unit 300 may rearrange the first final image signal in the direction from the center of the first K-space toward the peripheral side thereof as shown in the left drawing of FIG. 6, and the second final image signal in the direction from the peripheral side of the second K-space toward the center thereof as shown in the right drawing of FIG. 6. This rearrangement is based on the point that a signal of a low frequency area (around the center) in the K-space determines overall signal intensity of an image to be restored.

Specifically, the image signal receiving unit 300 rearranges the first final image signal in the direction from the center of the first K-space toward the peripheral side thereof such that the gray matter signal is highlighted more in a first reconstructed image to be restored from the first final image signal, which is described later. In case of the second final image signal, since the refocusing flip angles are set such that the signal intensities of cerebrospinal fluid are approximately the same between the first echo of the first echo train and the last echo of the second echo train, the image signal receiving unit 300 rearranges the second final image signal in the direction from the peripheral side of the second K-space toward the center thereof.

In addition, each of the image signals acquired from the two (2) consecutive echo trains may be subject to scattering sampling in a pseudo random manner in an elliptical K-space, and this method can reduce the number of times for repetition of the pulse train, and as a result, reduce the time required to acquire final images.

Returning to FIG. 3, the image generating unit 400 receives the first and second final image signals acquired in the above-described image signal receiving unit 300 to generate a selective gray matter image from the first and second final image signals. The image generating unit 400 may be provided in or combined to the image processing device 17.

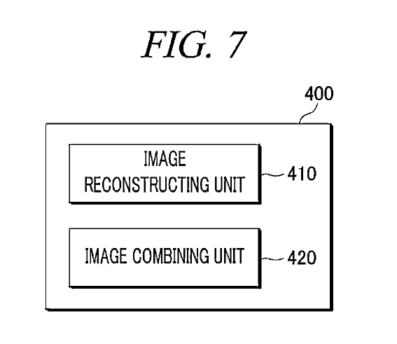

Detailed configuration of the image generating unit 400 is described with reference to FIG. 7. FIG. 7 is a block configuration diagram specifically showing the image generating unit of FIG. 3.

The image generating unit 400 includes an image reconstructing unit 410 that generates first and second reconstructed images from the first and second final image signals acquired in the image signal receiving unit 300, respectively, and an image combining unit 420 that implements weighted average processing for the first and second reconstructed images to generate a final selective gray matter image.

The image reconstructing unit 410 restores the first and second reconstructed images from the first and second final image signals, respectively, and various image restoration algorithms may be applied to the image reconstructing unit 410. As methods or algorithms applicable to the image reconstructing unit 410, there are Fourier transform, a multi-coil parallel imaging technique, a compressed sensing technique and so on.

The image combining unit 420 implements calculation of a weighting value for weighted averaging of the first and second reconstructed images, and eliminates the cerebrospinal fluid image signal so that a high-resolution selective gray matter image finally appears.

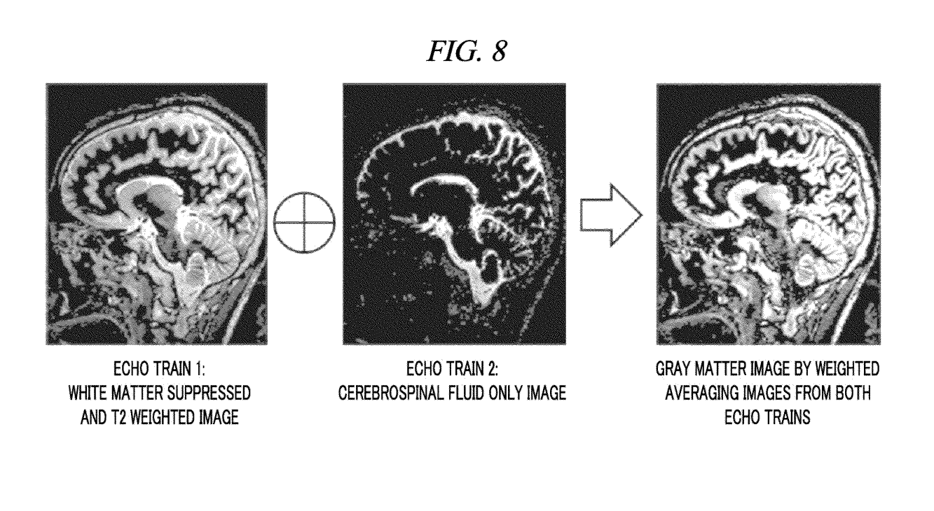

That is, with reference to FIG. 8 showing the selective gray matter image acquired by using the magnetic resonance imaging apparatus in accordance with an example embodiment, the first reconstructed image, in which a white matter signal is suppressed, and the second reconstructed image, in which white and gray matter signals are suppressed, are subject to weighted average processing so that a high-resolution gray matter image, from which the cerebrospinal fluid image signal is eliminated, can be obtained.

Further, as illustrated in FIG. 3, the magnetic resonance imaging apparatus in accordance with an example embodiment may further include a magnetization preparing unit 500 that prepares longitudinal magnetization prior to applying a first inversion pulse.

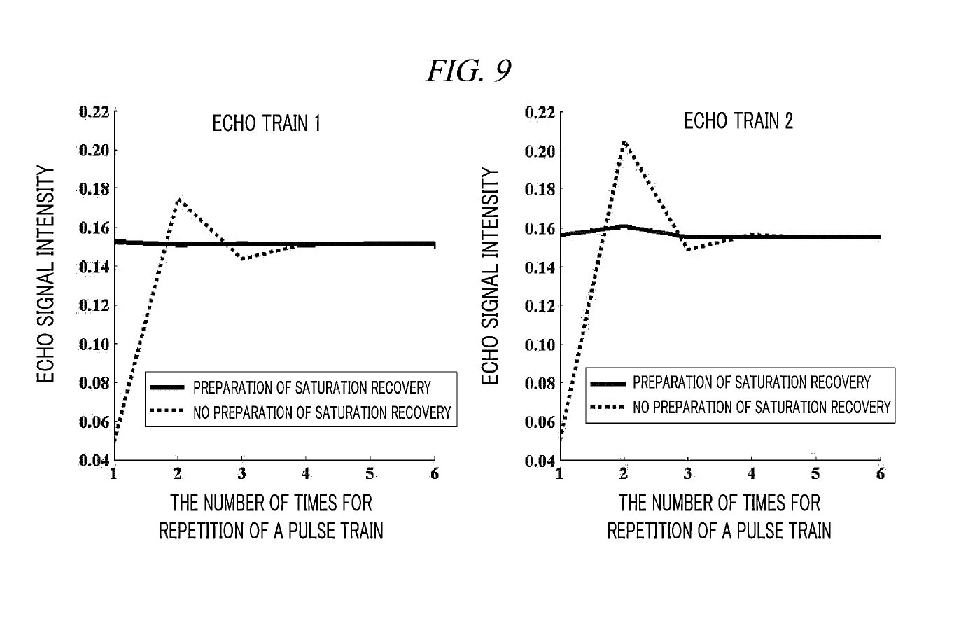

The magnetization preparing unit 500 is described with reference to FIG. 9. FIG. 9 shows a degree of change in signal intensities of the echo signal depending on whether or not magnetization preparation is applied.

If there is no magnetization preparing unit illustrated in FIG. 9, the intensity of the echo signal generated in each of the pulse trains varies with large width over initial several pulse trains until it reaches the steady state. This variation results in signal discontinuities among neighboring samples in K-space, potentially producing undesired artifacts in a restored image.

Accordingly, the magnetization preparing unit 500 generates and applies a pulse for preparation of longitudinal magnetization prior to the first pulse train for acquisition of data to enable the echo signal to rapidly reach the steady state, and insertion of the magnetization preparation pulse along with a period of time for magnetization recovery may occur only once prior to the application of the first inversion pulse.

When reviewing simulation results for change in the cerebrospinal fluid image signals of the first echoes in first echo trains and the last echoes in second echo trains in FIG. 9, it can be identified that the width of the variation of the echo signal is significantly large when no saturation recovery magnetization preparation is made, whereas the echo signal already enters into the steady state at first repetition of the pulse train when the saturation recovery magnetization preparation is made. In this case, the cerebrospinal fluid image signal may be mapped in the center of the K-space according to the repetition of the pulse train.

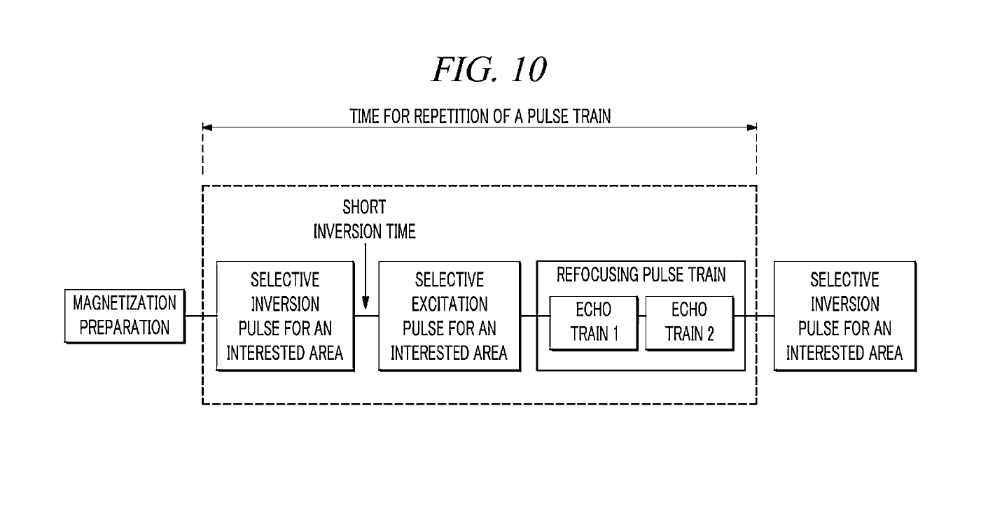

FIG. 10 shows the process for acquiring a selective gray matter image by using the magnetic resonance imaging apparatus in accordance with an example embodiment.

As shown in FIG. 10, the magnetization preparing unit 500 may operate before the repetition of the pulse train starts, and once the repetition of the pulse train starts, the inversion pulse generating unit 100, the excitation pulse generating unit 200, the image signal receiving unit 300, and the image generating unit 400 may begin to operate.

If the magnetic resonance imaging apparatus in accordance with an example embodiment that has been described is used, the time required to acquire the final image can be significantly reduced due to the application of only one inversion pulse with short inversion time, and since the final image signals are acquired from the two (2) consecutive echo trains, respectively, and independently processed, a selective gray matter image having a reduced signal to noise ratio loss and high resolution can be acquired.

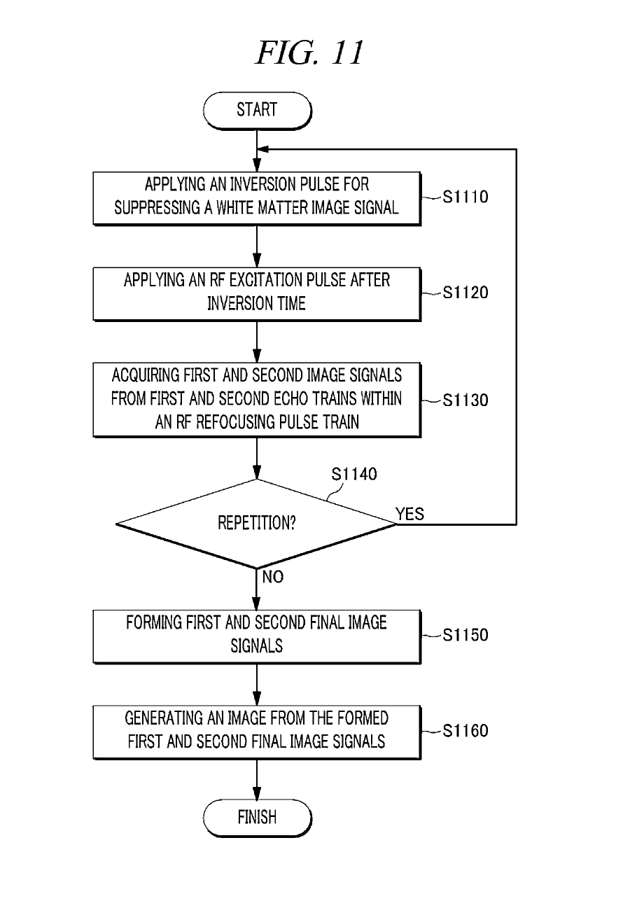

Meanwhile, the method for acquiring a magnetic resonance image in accordance with an example embodiment is described with reference to FIG. 11 to FIG. 13.

FIG. 11 is a sequence view showing the method for acquiring a magnetic resonance image in accordance with an example embodiment.

The method for acquiring an image in the magnetic resonance imaging apparatus in accordance with an example embodiment first applies the inversion pulse to a living body with short inversion time to suppress the white matter image signal (S1110).

That is, net magnetization of a living body tissue is in the state completely inversed toward (−) of the longitudinal axis by the 180° inversion pulse, and thereafter, a T1 relaxation phenomenon occurs according to a characteristic of each tissue so that magnetization recovers toward the (+) direction of the longitudinal axis.

During this process, a time point, at which the net magnetization in the direction of the longitudinal base of the tissue becomes zero (0), occurs, and the period of time from the time point of the application of the 180° inversion pulse to the time point, at which the net magnetization becomes zero (0), is referred to as inversion time (TI). For example, fat has inversion time of 150 ms, the white matter has inversion time of from 300 ms to 400 ms, the gray matter has inversion time of from 600 ms to 700 ms, and the cerebrospinal fluid has inversion time of from 2,000 m to 2,500 m.

Following the inversion pulse, the excitation pulse is applied after the inversion time of the tissue whose signal is sought to be suppressed. That is, the excitation pulse is applied after a delay about 300 ms to 400 ms from the 180° inversion pulse, so that the white matter image signal, which is a signal containing information related to the white matter image, can be suppressed.

As described above, magnetization is excited to the living body by applying the RF excitation pulse after the inversion time from the applied inversion pulse (S1120).

RF Refocusing pulses that constitute an RF refocusing pulse train are applied after the RF excitation pulse, and the first and second image signals are produced from the first and second echo trains within the RF refocusing pulse train, respectively (S1130).

This process is implemented over the time for the repetition of the pulse train as illustrated in FIG. 10, it is determined whether or not to repeat the process (S1140), and the process may be implemented once or more depending on a result of the determination.

If the process is implemented once, the first and second image signals may be decided as the first and second final image signals, and if the process is implemented twice or more, the first and second final image signals are formed by a multiple number of first and second image signals (S1150).

The image restoration algorithm is applied to each of the first and second final image signals that have been formed, and an additional processing process is implemented so that the final selective gray matter image is acquired (S1160).

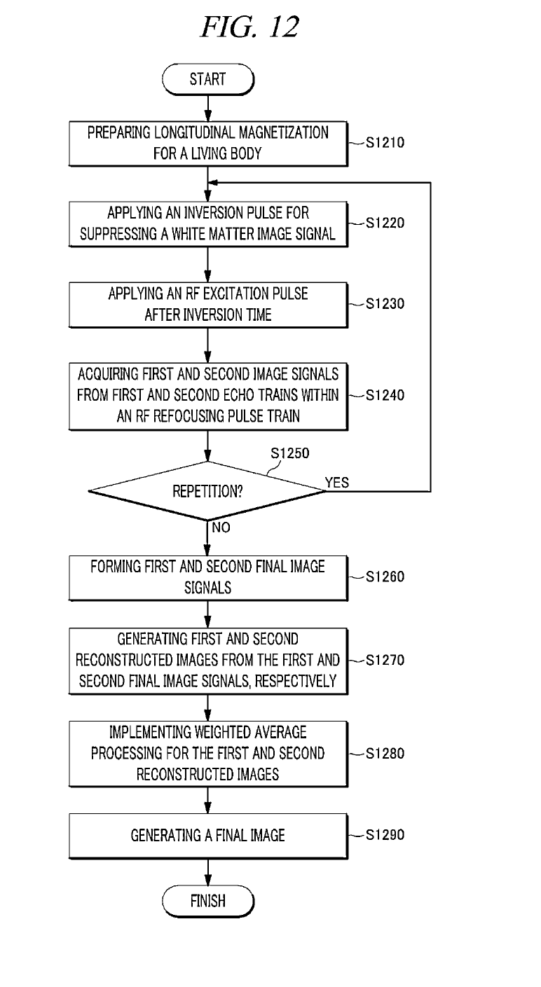

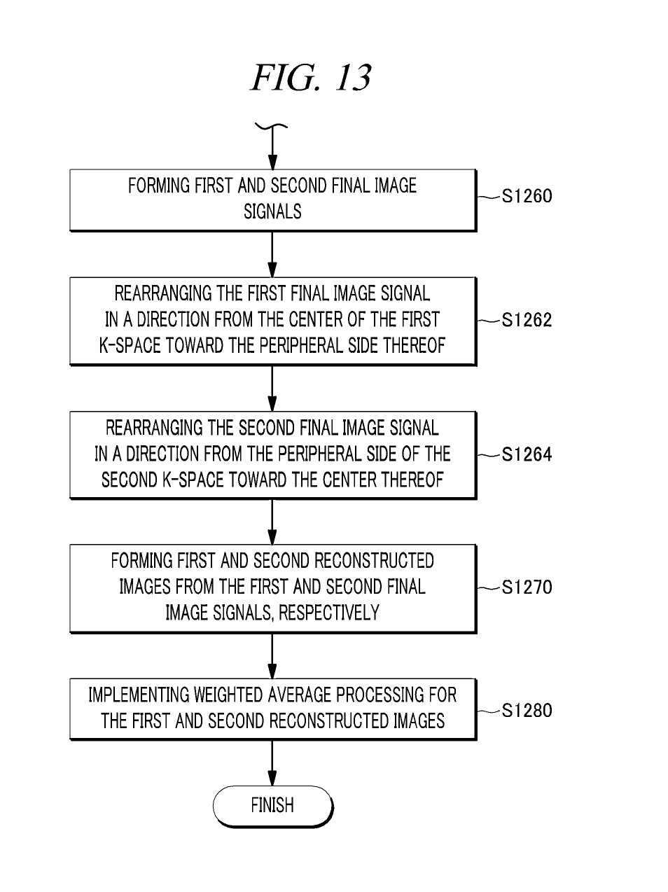

In addition, FIG. 12 is a sequence view showing a method for generating a magnetic resonance image in accordance with another example embodiment, and FIG. 13 is a sequence view specifically showing S1260 of FIG. 12.

First, longitudinal magnetization for the living body is prepared prior to an application of the first inversion pulse (S1210). Accordingly, the echo signal can stably enter into the steady state even at the time of the first repetition of the pulse train.

The inversion pulse is applied to an interested area of the living body with short inversion time so as to suppress the white matter image signal (S1220), and the RF excitation pulse is applied after the inversion time from the applied inversion pulse so as to excite magnetization to the living body (S1230).

After the application of the RF excitation pulse, a process for acquiring the first and second image signals from the first and second echo trains, respectively, within the RF refocusing pulse train is implemented (S1240).

This process may be implemented every time the pulse train is repeated, and a process for determining whether to repeat the process is implemented (S1250). If the process is implemented once more, the inversion pulse for suppressing the white matter image signal is applied without preparation of the longitudinal magnetization, and the follow-up process is repeated as described above. The first and second final image signals are decided from the first and second image signals acquired as a result of repetition of the process. If the process is not repeated, the first and second final image signals are immediately decided from the initially acquired first and second image signals.

Accordingly, the first and second final image signals are formed from implementing the series of processes that have been described once or multiple times (S1260).

In this case, the first final image signal may be rearranged in the direction from the center of the first K-space toward the peripheral side thereof (S1262), and the second final image signal may be rearranged in the direction from the peripheral side of the second K-space toward the center thereof (S1264).

Various image restoration algorithms are applied to the first and second final image signals that have been independently rearranged in the first and second K-spaces so that the first and second final image signals are restored into the first and second reconstructed images, respectively (S1270).

Each of the first and second reconstructed images that have been generated is subject to weighted average processing so that the final selective gray matter image is generated (S1280). That is, white matter image signal may not appear in the first reconstructed image, while both the gray and white matter image signals may not appear in the second reconstructed image. Once the first and second reconstructed images are subject to weighted average processing, the selective gray matter image, in which the cerebrospinal fluid image signal is suppressed, can be generated.

If the method for acquiring an image in the magnetic resonance imaging apparatus in accordance with an example embodiment is used, the time required to acquire the final image can be significantly reduced due to the application of only one inversion pulse with short inversion time, and since the final image signals are acquired from the two (2) consecutive echo trains, respectively, and independently processed, a selective gray matter image having a reduced signal to noise ratio loss and high resolution can be acquired.

The above description of the example embodiments is provided for the purpose of illustration, and it would be understood by those skilled in the art that various changes and modifications may be made without changing technical conception and essential features of the example embodiments. Thus, it is clear that the above-described example embodiments are illustrative in all aspects and do not limit the present disclosure. For example, each component described to be of a single type can be implemented in a distributed manner. Likewise, components described to be distributed can be implemented in a combined manner.

The scope of the inventive concept is defined by the following claims and their equivalents rather than by the detailed description of the example embodiments. It shall be understood that all modifications and embodiments conceived from the meaning and scope of the claims and their equivalents are included in the scope of the inventive concept.

We claim:

1. A magnetic resonance imaging apparatus comprising:
an inversion pulse generating unit that applies an inversion pulse to a living body to suppress a white matter image signal;
an excitation pulse generating unit that applies an RF excitation pulse to the living body after inversion time from the inversion pulse so as to excite magnetization;
an image signal receiving unit that acquires first and second final image signals from first and second echo trains in an RF refocusing pulse train, respectively; and
an image generating unit that generates a gray matter image from the first and second final image signals,
wherein the first and second final image signals are formed by first and second image signals acquired once or more from the first and second echo trains, respectively.

2. The magnetic resonance imaging apparatus of claim 1, further comprising a magnetization preparing unit that prepares longitudinal magnetization prior to an application of the first inversion pulse.

3. The magnetic resonance imaging apparatus of claim 1,
wherein the image generating unit comprises:
an image reconstructing unit that generates first and second reconstructed images from the first and second final image signals, respectively, and
an image combining unit that implements weighted average processing for the first and second reconstructed images to generate the gray matter image.

4. The magnetic resonance imaging apparatus of claim 1,
wherein the image signal receiving unit independently rearranges the first and second final image signals in first and second K-spaces, respectively,
the first final image signal is rearranged in the direction from the center of the first K-space toward the peripheral side thereof, and
the second final image signal is rearranged in the direction from the peripheral side of the second K-space toward the center thereof.

5. The magnetic resonance imaging apparatus of claim 1,
wherein the first final image signal comprises a gray matter image signal and a first cerebrospinal fluid image signal, and
the second final image signal comprises a second cerebrospinal fluid image signal.

6. The magnetic resonance imaging apparatus of claim 5,
wherein flip angles in the RF refocusing pulse train are set to make intensity of the first cerebrospinal fluid image signal and intensity of the second cerebrospinal fluid image signal identical to each other.

7. The magnetic resonance imaging apparatus of claim 5,
wherein flip angles in the RF refocusing pulse train are set to enable intensity of the gray matter image signal to have a predetermined value or more.

8. A method for acquiring a magnetic resonance image, comprising:
(a) applying an inversion pulse to a living body to suppress a white matter image signal;
(b) applying an RF excitation pulse to the living body after inversion time from the inversion pulse to excite magnetization;
(c) acquiring first and second image signals from first and second echo trains within an RF refocusing pulse train, respectively;
(d) implementing steps (a) to (c) above once or more to form first and second final image signals by the first and second image signals; and
(e) acquiring a gray matter image from the first and second final image signals.

9. The method for acquiring a magnetic resonance image of claim 8, further comprising preparing longitudinal magnetization in the living body prior to the application of the first inversion pulse in the step (a) above.

10. The method for acquiring a magnetic resonance image of claim 8,
wherein the first and second final image signals are independently rearranged in first and second K-spaces, respectively.

11. The method for acquiring a magnetic resonance image of claim 10,
wherein the first final image signal is rearranged in the direction from the center of the first K-space toward the peripheral side thereof, and
the second final image signal is rearranged in the direction from the peripheral side of the second K-space toward the center thereof.

12. The method for acquiring a magnetic resonance image of claim 8,
wherein the step (e) above comprises:
generating first and second reconstructed images from the first and second final image signals; and
implementing weighted average processing for the first and second reconstructed images to generate the gray matter image.
